(19) United States
(12) Patent Application Publication
(10) Pub. No.: US 2023/0181278 A1
(43) Pub. Date: Jun. 15, 2023

(54) PROTECTIVE BARRIERS FOR MEDICAL USE

(71) Applicant: Redsmith, Inc., Walnut Creek, CA (US)

(72) Inventors: James D. Mitchell, Walnut Creek, CA (US); Andrew Thoreson, Orono, MN (US); Jeanne Sue Pae, Walnut Creek, CA (US)

(21) Appl. No.: 17/906,690

(22) PCT Filed: Mar. 20, 2021
(86) PCT No.: PCT/US21/23365

§ 371 Date: Sep. 19, 2022

Related U.S. Application Data
(60) Provisional application No. 63/011,236, filed on Apr. 16, 2020.
(60) Provisional application No. 62/994,842, filed on Mar. 26, 2020.
(60) Provisional application No. 62/992,712, filed on Mar. 20, 2020.

(51) Int. Cl. A61B 46/20

(52) U.S. Cl. CPC A61B 46/20; A61B 2046/205

(57) ABSTRACT

Devices, systems, and methods for protecting medical personnel during medical procedures are disclosed herein. The present technology may comprise, for example, a flexible barrier configured to be positioned over all or a portion of the patient's head during the procedure. The barrier may comprise a first side configured to face towards the patient during the procedure and a second side configured to face towards a healthcare worker during the procedure. The barrier can include an access region configured to be positioned at or near a mouth of the patient when the barrier is positioned on the patient during the procedure. The access region can be configured to provide passage of a medical device through the barrier and into an oral cavity of the patient while maintaining a physical barrier around the medical device.

### CROSS-REFERENCE TO RELATED APPLICATION(S)

The present application claims the benefit of priority of U.S. Provisional Application No. 62/992,712, filed Mar. 20, 2020, U.S. Provisional Application No. 62/994,842, filed Mar. 26, 2020, and U.S. Provisional Application No. 63/011,236, filed Apr. 16, 2020, all of which are incorporated by reference herein in their entireties.

### TECHNICAL FIELD

The present technology relates to a protective barrier for medical applications. In particular embodiments, the present technology relates to a protective barrier for use during endotracheal intubation and other medical procedures.

### BACKGROUND

Respiratory pathogens can include viruses, bacteria, fungi, protozoa, helminths, and other parasitic organisms. Upper and lower respiratory viruses and bacteria are especially virulent, transmitting from person to person via respiratory droplets, aerosolized secretions, or airborne pathogens. Common sources of respiratory infection include rhinovirus, coronavirus, influenza, streptococcus pneumoniae, streptococcus pyogenes, mycobacterial organisms like tuberculosis, and haemophilus influenza. The coronavirus disease 2019 (COVID-19) represents an especially virulent respiratory pathogen with high rates of transmission, infection, and death, similar to the viruses that cause severe acute respiratory syndrome (SARS) and Middle East respiratory syndrome (MERS).

Any patient infected with a respiratory pathogen represents a risk to healthcare workers. Because of the ease of transmission, healthcare workers must use personal protective equipment (PPE) in order to prevent exposure to these common infections. PPE often includes gloves, masks (such as surgical masks or N95 masks), eye shields, and gowns. However, PPE does not provide full protection, as infection is possible despite correct use of PPE. This has been seen during the current COVID-19 pandemic and other outbreaks, such as recent Ebola outbreaks and others.

Endotracheal intubation is frequently required when patients present in respiratory distress. As shown in FIG. 1, the procedure involves placement of an endotracheal tube through the patient's oral cavity and pharynx and into the trachea. The endotracheal tube secures an open airway and allows a conduit for mechanical ventilation. As shown in FIG. 2, intubation is normally facilitated by using a laryngoscope to identify the vocal cords and pass the tube between the vocal cords into the trachea (instead of into the esophagus). An example of a commercially-available fiberoptic laryngoscope is the Glidescope® system (Verathon, Inc., Bothell, Wash.), shown in FIG. 3.

During an intubation procedure, the patient's upper airway remains open and upper airway pathogens are released into the surrounding environment. Because the clinician performing the procedure is necessarily standing at the patient's head, the clinician is particularly susceptible to exposure to infectious organisms expelled into the environment, including secretions and aerosolized and airborne microbes. Therefore, there is a need for improved protective barriers for use during endotracheal intubations and other medical procedures.

### SUMMARY

The present technology is directed to a medical barrier to prevent or substantially inhibit the spread of pathogens during medical procedures. Specifically, the technology is intended as a barrier to prevent or substantially inhibit transmission of respiratory pathogens from a patient to a healthcare worker or from a patient to another patient. The subject technology is illustrated, for example, according to various aspects described below, including with reference to FIGS. 1-37.
1. A device for preventing or substantially inhibiting the dissemination of pathogens from a patient to a surrounding environment during a medical procedure, the device comprising:
a flexible barrier configured to be positioned over all or a portion of the patient's face during the procedure, the barrier comprising—
   a broad portion having a first side configured to face towards the patient during the procedure and a second side configured to face towards a healthcare worker during the procedure, wherein the broad portion includes first and second access regions, each configured to receive a medical device therethrough, and wherein the first and second access regions are configured to be positioned at or near a mouth of the patient when the barrier is positioned on the patient during the procedure;
   a tubular portion extending from the second side of the broad portion, the tubular portion configured to receive an endotracheal access device therethrough.
2. The device of any one of the preceding Clauses, wherein the tubular portion has a proximal end portion at the broad portion and a distal end portion opposite the proximal end portion along a length of the tubular portion, and wherein:
the proximal end portion includes a proximal opening configured to receive a portion of the endotracheal access device therethrough, and
the distal end portion includes a distal opening configured to receive one or more cables associated with the endotracheal access device therethrough.
3. The device of Clause 2, wherein the proximal end portion of the tubular portion comprises a valve.
4. The device of any one of the preceding Clauses, wherein the endotracheal access device is a laryngoscope.
5. The device of any one of the preceding Clauses, wherein the proximal opening of the tubular portion is configured to receive a blade of a laryngoscope therethrough.
6. The device of any one of the preceding Clauses, wherein the tubular portion is configured to receive a laryngoscope handle therethrough.
7. The device of any one of the preceding Clauses, wherein the tubular portion is predisposed at an acute angle relative to the second side of the broad portion.
8. The device of any one of the preceding Clauses, wherein the tubular portion meets the broad portion at a flexible joint configured to allow the healthcare worker to change an angle of the tubular portion relative to the broad portion.
9. The device of any one of the preceding Clauses, wherein the broad portion is sized to cover all or a portion of an upper torso of the patient and a head of the patient.
10. The device of any one of the preceding Clauses, further comprising an extension extending from an edge of the broad portion, wherein the extension is sized to cover all or a portion of an upper torso of the patient and a head of the patient.
11. The device of Clause 10, wherein the extension comprises an adhesive at its patient-facing surface configured to adhere to the patient's body.
12. The device of any one of the preceding Clauses, wherein the first and/or second access regions are configured to receive at least one of an endotracheal tube or a suction device therethrough.
13. The device of any one of the preceding Clauses, wherein the first and/or second access regions comprise slits in the broad portion.
14. The device of any one of the preceding Clauses, wherein the first and/or second access regions comprise a single slit in the broad portion.
15. The device of any one of the preceding Clauses, wherein the first and/or second access regions comprise a plurality of slits arranged in an “x”, star, or pinwheel shape.
16. The device of any one of the preceding Clauses, wherein the first and/or second access regions comprise a plurality of linear and/or curved slits radiating outwardly from a central point.
17. The device of any one of the preceding Clauses, wherein the first and/or second access regions include a valve.
18. The device of any one of the preceding Clauses, wherein the first and/or second access regions include a bicuspid valve.
19. The device of any one of the preceding Clauses, wherein the first and/or second access regions include a valve configured to open in response to insertion of an endotracheal tube or suction device, and close once the endotracheal tube or suction device is withdrawn.
20. The device of any one of the preceding Clauses, wherein the first and/or second access regions include a spring-activated hinge valve.
21. The device of any one of the preceding Clauses, wherein the first and second access regions are configured to provide passage of a medical device through the barrier and into an oral cavity of the patient while maintaining a physical barrier around the medical device.
22. The device of any one of the preceding Clauses, wherein the broad portion further comprises a third access region configured to receive at least one of an endotracheal tube and/or a suction device therethrough.
23. The device of Clause 22, wherein the broad portion further comprises a fourth access region configured to receive a medical instrument therethrough.
24. The device of any one of the preceding Clauses, wherein the barrier is disposable.
25. The device of any one of the preceding Clauses, wherein the barrier is configured for single use.
26. The device of any one of the preceding Clauses, wherein all or a portion of the broad portion is translucent.
27. The device of any one of the preceding Clauses, wherein all or a portion of the broad portion is transparent.
28. The device of any one of the preceding Clauses, wherein at least a portion of the barrier is translucent.
29. The device of any one of the preceding Clauses, wherein at least a portion of the barrier is transparent.
30. The device of any one of the preceding Clauses, wherein all of the barrier is translucent.
31. The device of any one of the preceding Clauses, wherein all of the barrier is transparent.
32. The device of any one of the preceding Clauses, wherein the barrier comprises an antifog coating.
33. The device of any one of the preceding Clauses, wherein the barrier comprises an adhesive along all or a portion of a surface of the first side of the broad portion, the adhesive configured to (a) detachably adhere to the patient's skin and/or (b) detachably adhere to a surface in the vicinity of the patient.
34. The device of any one of the preceding Clauses, wherein the barrier comprises an adhesive along selected portions of a surface of the first side of the broad portion, wherein, wherein the barrier is positioned on the patient's head during the procedure, the selected portions correspond to one or more of the patient's nose, the patient's cheeks, and/or a portion of the patient's head inferior to a chin of the patient.
35. The device of any one of the preceding Clauses, wherein the broad portion and the tubular portion are formed of the same, continuous piece of material.
36. A device for preventing or substantially inhibiting the dissemination of pathogens from a patient to the surrounding environment during endotracheal intubation of the patient, the device comprising:
a flexible barrier configured to be positioned over all or a portion of the patient's head during the procedure, the barrier comprising a first side configured to face towards the patient during the procedure and a second side configured to face towards a healthcare worker during the procedure, wherein the barrier includes an access region configured to be positioned at or near a mouth of the patient when the barrier is positioned on the patient during the procedure, and wherein the access region is configured to provide passage of a medical device through the barrier and into an oral cavity of the patient while maintaining a physical barrier around the medical device.
37. A device for preventing or substantially inhibiting the dissemination of pathogens from a patient to the surrounding environment during endotracheal intubation of the patient, the device comprising:
a flexible barrier configured to be positioned over all or a portion of the patient's head during the procedure, the barrier comprising a first side configured to face towards the patient during the procedure and a second side configured to face towards a healthcare worker during the procedure, wherein the barrier includes first and second access regions configured to be positioned at or near a mouth of the patient when the barrier is positioned on the patient during the procedure, and wherein each of the first and second access regions are configured to provide passage of a medical device through the barrier and into an oral cavity of the patient while maintaining a physical barrier around the medical device.
38. The device of any one of the preceding Clauses, wherein the barrier is sized to cover all or a portion of an upper torso of the patient and a head of the patient.
39. The device of any one of the preceding Clauses, further comprising an extension extending from an edge of the barrier wherein the extension is sized to cover all or a portion of an upper torso of the patient and a head of the patient.
40. The device of Clause 38, wherein the extension comprises an adhesive at its patient-facing surface configured to adhere to the patient's body.
41. The device of any one of the preceding Clauses, wherein the first and/or second access regions are configured to receive at least one of an endotracheal tube, a laryngoscope, an endoscope, or a suction device therethrough.
42. The device of any one of the preceding Clauses, wherein the first and/or second access regions comprise slits in the barrier.
43. The device of any one of the preceding Clauses, wherein the first and/or second access regions comprise a single slit in the barrier.
44. The device of any one of the preceding Clauses, wherein the first and/or second access regions comprise a plurality of slits arranged in an “x”, star, or pinwheel shape.
45. The device of any one of the preceding Clauses, wherein the first and/or second access regions comprise a plurality of linear and/or curved slits radiating outwardly from a central point.
46. The device of any one of the preceding Clauses, wherein the first and/or second access regions include a valve.
47. The device of any one of the preceding Clauses, wherein the first and/or second access regions include a bicuspid valve.
48. The device of any one of the preceding Clauses, wherein the first and/or second access regions include a valve configured to open in response to insertion of an endotracheal tube or suction device, and close once the endotracheal tube or suction device is withdrawn.
49. The device of any one of the preceding Clauses, wherein the first and/or second access regions include a spring-activated hinge valve.
50. The device of any one of the preceding Clauses, wherein the barrier further comprises a third access region configured to receive at least one of an endotracheal tube and/or a suction device therethrough.
51. The device of Clause 49, wherein the broad portion further comprises a fourth access region configured to receive a medical instrument therethrough.
52. The device of any one of the preceding Clauses, wherein the barrier is disposable.
53. The device of any one of the preceding Clauses, wherein the barrier is configured for single use.
54. The device of any one of the preceding Clauses, wherein at least a portion of the barrier is translucent.
55. The device of any one of the preceding Clauses, wherein at least a portion of the barrier is transparent.
56. The device of any one of the preceding Clauses, wherein all of the barrier is translucent.
57. The device of any one of the preceding Clauses, wherein all of the barrier is transparent.
58. The device of any one of the preceding Clauses, wherein the barrier comprises an antifog coating.
59. The device of any one of the preceding Clauses, wherein the barrier comprises an adhesive along all or a portion of a surface of the first side, the adhesive configured to (a) detachably adhere to the patient's skin and/or (b) detachably adhere to a surface in the vicinity of the patient.
60. The device of any one of the preceding Clauses, wherein the barrier comprises an adhesive along selected portions of a surface of the first side, wherein, wherein the barrier is positioned on the patient's head during the procedure, the selected portions correspond to one or more of the patient's nose, the patient's cheeks, and/or a portion of the patient's head inferior to a chin of the patient.
61. The device of any one of the preceding Clauses, wherein the barrier comprises a reinforced portion at the access region, wherein the reinforced portion has a thickness greater than a thickness of the surrounding portions of the barrier.
62. The device of any one of the preceding Clauses, wherein the barrier comprises a reinforced portion at the access region, wherein the reinforced portion is less flexible than the rest of the barrier.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows an endotracheal tube of the prior art inserted into the airway and the relationship to the relevant anatomy of the oral cavity, pharynx, and larynx.

FIG. 2 shows a laryngoscope of the prior art positioned in the patient's oral cavity and pharynx to assist placement of an endotracheal tube of the prior art in the patient's airway.

FIG. 3 shows an example laryngoscope of the prior art.

FIG. 4 is a top view of a barrier configured in accordance with several embodiments of the present technology.

FIG. 5 is a top view of the access region of the barrier shown in FIG. 4, shown isolated from the barrier.

FIG. 6 is an isometric view of the access region of the barrier shown in FIG. 4, shown isolated from the barrier.

FIG. 7A depicts a barrier configured in accordance with embodiments of the present technology positioned on a patient with an access region aligned with the patient's oral cavity.

FIG. 7B is another view of the barrier shown in FIG. 7A.

FIGS. 7C-7E depict a barrier configured in accordance with embodiments of the present technology positioned on a patient and receiving multiple instruments therethrough during a medical procedure.

FIG. 8 is a top view of a barrier configured in accordance with several embodiments of the present technology.

FIG. 9 is a top view of a barrier in accordance with several embodiments of the present technology showing another spatial arrangement access regions for instrument passage.

FIG. 10 is a side view of a barrier configured in accordance with several embodiments of the present technology.

FIG. 11 is a side view of a barrier configured in accordance with several embodiments of the present technology wherein the tubular portion is predisposed at an angle relative to the broad portion to accommodate the shape of the laryngoscope.

FIG. 12 is a side view of a barrier configured in accordance with several embodiments of the present technology demonstrating a sleeve type cover for the laryngoscope handle with the laryngoscope positioned within said sleeve.

FIG. 13 is a side perspective view demonstrating midline anatomy during an intubation procedure. FIG. 13 depicts a sleeve type cover to the laryngoscope device and a barrier drape covering the nose and mouth of the patient.

FIG. 14 is an en face view of the barrier shown in FIG. 13.

FIG. 15 is a side view of the device shown in FIG. 12 demonstrating an aperture for passage of an electrical and fiberoptic cable to the handle of the laryngoscope.

FIG. 16 is a side view with midline patient anatomy demonstrating an endotracheal tube inserted into the trachea with the barrier device still in place covering the patient's nose and mouth.

FIG. 17 shows the same device as FIG. 16, but demonstrating a break away cover for the laryngoscope which can be removed at the completion of the procedure.

FIG. 18 is a side view of the device shown in FIG. 10, but with an additional drape to cover the entire head and upper torso of the patient.

FIG. 19 is a side view of the device shown in FIG. 16, but with an additional drape to cover the entire head and upper torso of the patient.

FIG. 20 is a side view of the device shown in FIG. 14, but with an additional drape to cover the entire head and upper torso of the patient.

FIG. 21 is a top view of the preferred embodiment demonstrating a drape large enough to cover the entire head and upper torso of the patient with adhesive along the edges. Four passages are arranged over the mouth for insertion of medical instrumentation.

FIG. 22 is an enlarged top view detailed image of the embodiment described in FIG. 21 with exemplary orientation and dimensions.

FIG. 23 is an enlarged view of the embodiment shown in FIG. 22 with an optional additional flap to provide barrier protection over the passages for instrumentation.

FIG. 24 is a top view of a barrier configured in accordance with embodiments of the present technology, shown with optional labeling of the passages to orient the user.

FIGS. 25A-2D show various valves of a barrier configured in accordance with several embodiments of the present technology.

FIG. 26A is a top view and cross section view showing possible orientations of valves used in some embodiments.

FIG. 26B demonstrates possible elimination of one of the instrument passages and/or valves. FIG. 26B is an enlarged view of the configuration if using bicuspid valves.

FIG. 27 is a top view of a portion of a barrier configured in accordance with several embodiments of the present technology.

FIG. 28 is a top view of a barrier configured in accordance with several embodiments of the present technology.

FIG. 29 is a top view of a barrier configured in accordance with several embodiments of the present technology.

FIGS. 30A and 30B are top and side views of a barrier configured in accordance with several embodiments of the present technology.

FIG. 31 depicts a barrier configured in accordance with several embodiments of the present technology positioned on a patient's head prior to any instrumentation being inserted through the barrier.

FIG. 32 shows a barrier configured in accordance with several embodiments of the present technology positioned on a patient's head with a medical instrument positioned through the barrier and into the patient's oral cavity and trachea.

FIG. 33 is an access region configured in accordance with several embodiments of the present technology.

FIG. 34A is a top view of the access region shown in FIG. 33, shown with a medical instrument inserted therethrough.

FIG. 34B is a cross-sectional side view of the access region shown in FIG. 33, shown with a medical instrument inserted therethrough.

FIG. 35 is a top view of a barrier configured in accordance with several embodiments of the present technology.

FIG. 36 is an end view of a barrier configured in accordance with several embodiments of the present technology. In FIG. 36, the barrier is shown positioned over a patient.

FIG. 37 is a schematic illustration of a barrier configured in accordance with embodiments of the present technology.

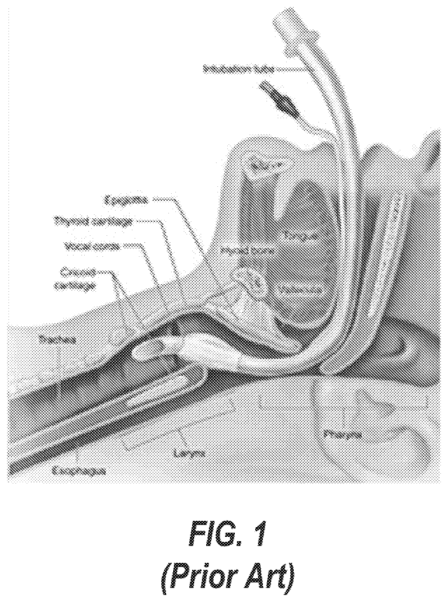

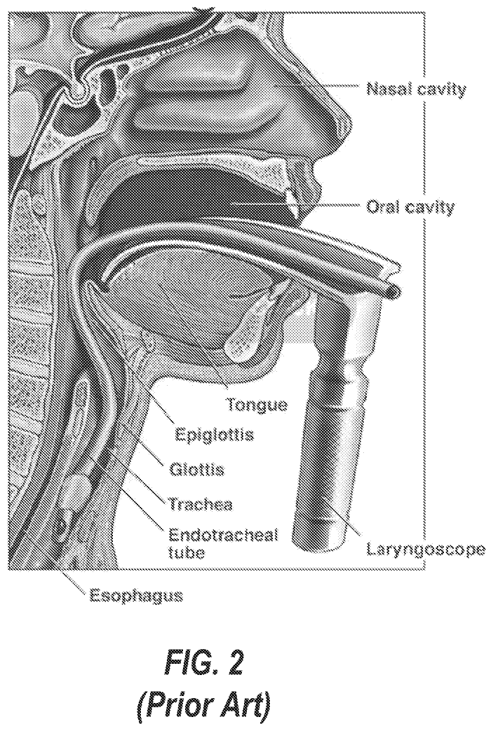

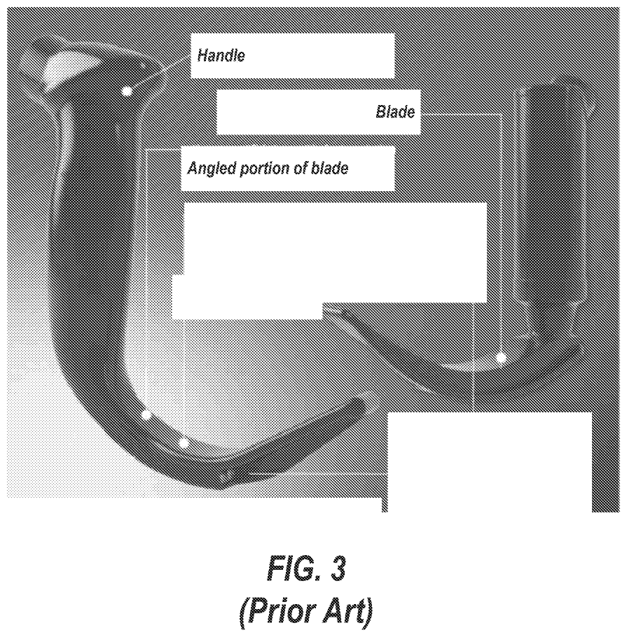

### DETAILED DESCRIPTION

The present technology comprises a barrier configured to be positioned on or near a patient to protect healthcare professionals during medical procedures requiring extended access to the patient's airways, such as endotracheal intubations, endoscopies, bronchoscopies, and others. Currently, no devices or products exist for this purpose. The barrier is configured to cover all or a portion of the patient's nose and mouth during the procedure, as well as cover all or a portion of the medical device(s) used in the procedure, such as a laryngoscope or other tracheal access device. In some embodiments, the barrier comprises a material having one or more apertures, each configured to receive one or more medical devices therethrough. For example, according to some embodiments, the barrier includes a first aperture to allow for electrical and fiberoptic connection through the barrier, and a second aperture for passage of an endotracheal tube and suction equipment. A second aperture may also be included to allow for suctioning while the endotracheal tube is being passed through the barrier. The barrier may optionally include a third aperture for additional instrumentation or to account for left-handed operators.

According to some embodiments of the present technology, the barrier is made of a clear, flexible material that allows for manipulation and motion of the medical devices used during the procedure, such as a laryngoscope, endotracheal tube, and suction equipment (e.g., a Yankauer suction tip). The surface of the barrier device that faces the patient preferably has a medical grade, hypoallergenic adhesive to provide a seal between the barrier and the patient's skin, or to fix the edges of the barrier in place along the bed, stretcher, operating room table, and/or the patient's skin. The barrier could be stuck to the skin of the patient's face such that a seal is produced along the bridge of the nose (superior to the nostrils), along both cheeks lateral to the angles of the mouth, and below (inferior to) the chin. In some embodiments, the barrier does not include any adhesive and is configured to be loosely draped over the patient.

In some embodiments, the barrier described herein may be transparent to allow the medical professional to visualize the path of the laryngoscope, and thus the patient's larynx for insertion of the tube. This is less important for those using a fiberoptic laryngoscope, but visualization of the anatomy is always preferred. The barrier would preferably have an antifog coating to prevent opacification of the barrier from water vapor in the patient's respiratory tract.

The barriers of the present technology may be configured to allow passage of the endotracheal tube through the barrier into the patient's airway, as well as to allow suctioning of secretions from the patient's upper airway and oral cavity. An aperture may be placed adjacent to the cover for the laryngoscope handle. In some embodiments, this aperture contains a valve that is by default in the closed position. The valve then opens as a result of physical pressure exerted with insertion of the endotracheal tube or suction device, and the closes again with removal of the tube or suction device. An example of such a valve includes a spring activated hinge valve. A second aperture and valve combination could be included either adjacent to the first aperture or on the opposite side of the laryngoscope handle cover. The second aperture would provide a means of passing a suction tip into the upper airway at the same time as the endotracheal tube.

The barriers of the present technology described herein may include a tubular member for covering the handle of the laryngoscope. The cover allows the operator to manipulate the laryngoscope device while still being protected by the barrier. The most proximal portion of the handle cover may include an aperture to allow passage of electrical and fiber optic cables for use with more advanced fiber optic laryngoscopes.

In some embodiments of the barrier described herein, the device comprises a drape that covers the upper torso and entire head of the patient. Adhesive may be added along the edges of the drape to maintain positioning with respect to the patient, and additional adhesive may allow affixing of the barrier drape to any other part of the patient's anatomy. In the central portion of the barrier are 4 passages to allow delivery of medical instrumentation into the patient's oral cavity. The passages are comprised of precut or perforated slits in the shape of an “x”, star, pinwheel, or other array of lines and/or arcs radiating out from a central point. If the top of the patient's head is considered the 12:00 position, then the passage in the 6:00 position (inferior position anatomically) is largest and designed for passage of a laryngoscope during intubation. Three smaller, yet similar in size to each other, passages are arranged in approximately the 12:00, 10:30 and 1:30 positions to allow for passage of additional instrumentation such as endotracheal tubes, suction probes such as Yankauer devices, or other medical devices such as bronchoscopes or endoscopes. In some embodiments, one of the three passages could be eliminated assuming all medical professionals perform said procedures with the same handedness. The “leaves” of each passage remain in the closed position due to the material's natural rigidity and memory. In these any embodiment described herein, the “leaves” may comprise medical grade silicone, 30 durometer with “memory” properties such that the aperture leaves return to the original closed position when instrumentation is retracted. When a medical instrument is introduced through the passage, the leaves of the passage are pushed to the side to create an aperture for the instrument while still providing a physical barrier around the instrument. When said instrument is removed, the leaves return to the resting position due to the material's physical memory, maintaining a physical barrier between the patient's upper respiratory tract and the medical professional performing the procedure. The endpoint of each slit or perforation radiating outward from the center of the instrument passage is reinforced to prevent propagation of a tear into the surrounding material. This reinforcement can be based on shape with a cul-de-sac end to the cut or perforation, stitching, or any form of physical reinforcing member.

In some embodiments, the passages described above carry valves to promote both insertion and removal of medical instruments during a procedure. The valves are shaped as bicuspid leaves made of a pliable material with physical memory such as medical grade silicone. With insertion of a medical instrument into the valve, the leaves of the valve are displaced downward and to the side creating a partial seal around the instrument. Removal of the instrument displaces the valve leaves in the opposite direction. Upon complete removal of the instrument, the bicuspid leaves return to their resting position, maintaining a physical barrier.

The barriers of the present technology may be configured for a single use, disposable medical device. Once the endotracheal tube is securely in place, the barrier device can be removed by sliding the aperture over the proximal aspect of the tube, or preferably the aperture can break away and be peeled off the tube as to not disrupt its position within the patient. Additionally, the handle cover can either break away, or the proximal aperture can allow passage of the laryngoscope after the tube is secured. Finally, if preferred, once the handle cover is broken away and the laryngoscope is removed, the opening left behind could be covered with an additional barrier patch and left in place to provide barrier protection during mechanical ventilation.

The barrier may be configured to be positioned at, on, or over all or a portion of a head of a patient during intubation of the patient. The barrier may comprise a flexible material configured to prevent or substantially inhibit dissemination of pathogens from the patient to the surrounding environment. According to some embodiments, the barrier 100 may comprise a broad portion and a tubular portion.

The broad portion may have a first side configured to face towards the patient during the procedure and a second side configured to face towards a healthcare worker during the procedure. The broad portion may include first and second access regions, each configured to receive a medical device therethrough, such as an endotracheal tube or a suction device. The tubular portion and the first and second access regions are disposed on the broad portion such that, when the barrier is positioned on a patient during intubation, a proximal end of the tubular portion and the first and second access regions are positioned at or near a mouth of the patient. Each of the first and second access regions are configured to provide passage of a medical device through the barrier and into an oral cavity of the patient while maintaining a physical barrier around the medical device extending therethrough.

The tubular portion may extend from the first side of the broad portion. The tubular portion may have a lumen configured to receive an endotracheal access device therethrough. The tubular portion may have a proximal end portion at the broad portion and a distal end portion opposite the proximal end portion along a length of the tubular portion. According to some embodiments, the proximal end portion of the tubular portion includes a proximal opening configured to receive a portion of the endotracheal access device therethrough (such as a laryngoscope), and the distal end portion includes a distal opening configured to receive one or more cables associated with the endotracheal access device therethrough. In some embodiments, the proximal end portion of the tubular portion comprises a valve.

In some embodiments, for example as shown in FIG. 11, the tubular portion is predisposed at an acute angle relative to the broad portion to accommodate the shape of a laryngoscope. In some embodiments, the tubular portion is positioned substantially perpendicular to the broad portion. The proximal opening at the proximal end portion of the tubular portion (e.g., where the tubular portion meets the broad portion) may be configured to receive a blade of a laryngoscope therethrough. Moreover, the tubular portion may be configured to receive a laryngoscope handle therethrough.

In some embodiments, the broad portion and the tubular portion are formed of the same, continuous material. In some embodiments, the broad portion and the tubular portion are separate components that are coupled together.

Any of the barriers disclosed herein may comprise a flexible member including a layer of a stiffer, more resilient material (but still elastic) over the passages and/or access regions. Such a material may include silicone, a hydrogel, and others.

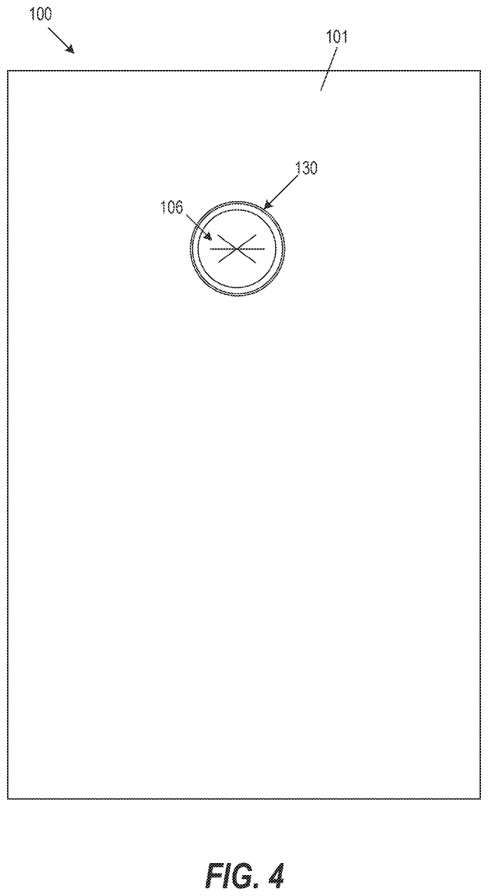

FIG. 4 is a top view of a barrier 100 configured in accordance with several embodiments of the present technology. The barrier 100 may be configured to be positioned at, on, or over all or a portion of a head of a patient during intubation of the patient. In some embodiments, the barrier 100 may be positioned over additional portions of the patient's body, such as the patient's torso. The barrier 100 may comprise a flexible member 101 configured to prevent or substantially inhibit dissemination of pathogens from the patient to the surrounding environment. The barrier 100, for example, may be made of medical grade silicone, a polymer, and other materials. The barrier 100 can have a first side configured to face towards a healthcare worker during the procedure and a second side configured to face towards the patient during the procedure. Each of the first and second sides have first and second surfaces, respectively. All or a portion of the second surface may include an adhesive configured to detachably adhere to the patient's skin or garments, or a nearby structure in the patient's environment (such as the bed, a side rail, etc.). In some embodiments, the second surface does not include any adhesive.

According to several embodiments of the present technology, all or a portion of the barrier 100 is translucent or transparent so that a healthcare worker can see the patient during the procedure.

The barrier 100 may include one or more access regions 106 configured to receive a medical device therethrough, such as an endotracheal access device (such as laryngoscope), an endotracheal tube, a suction device, and/or other devices described herein. The access regions 106 may be disposed on the barrier 100 such that, when the barrier 100 is positioned on a patient during intubation, the access regions 106 are positioned at or near a mouth of the patient. Each of the access regions 106 may be configured to provide passage of a medical device through the barrier 100 and into an oral cavity of the patient while maintaining a seal around the medical device extending therethrough.

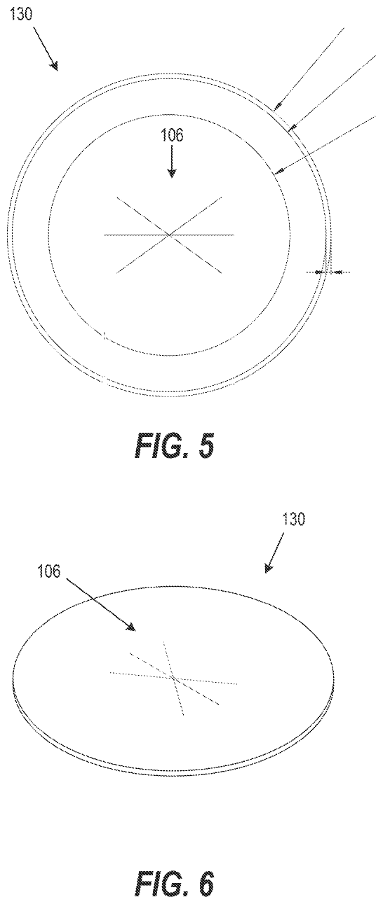

In some embodiments, the access regions 106 may be disposed at a reinforced portion 130 of the barrier 100. For example, the reinforced portion 130 of the barrier 100 may have a stiffness that is greater than that of the barrier 100. The reinforced portion 130 may comprise the same or different material as the member 101. In some embodiments, the reinforced portion 130 has an average thickness that is greater than an average thickness of the member 101. FIG. 5 is a top view of the reinforced portion 130 of the barrier shown in FIG. 4, shown isolated from the barrier 100. FIG. 6 is an isometric view of the reinforced portion of the barrier 100 shown in FIG. 4, shown isolated from the barrier 100.

According to some embodiments, for example as shown in FIGS. 4-6, one or more of the access regions 106 may comprise one or more slits in the flexible member comprising the barrier 100. Each of the slits may meet at an intersection. The slits may define flaps or leaflets 200 therebetween that also meet at the intersection. These and other access region configurations are described in greater detail herein.

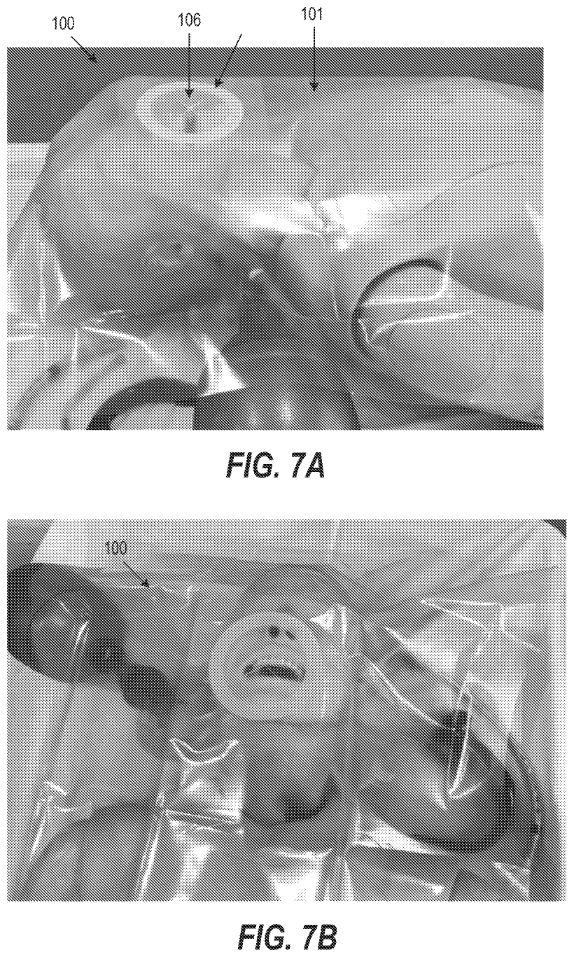

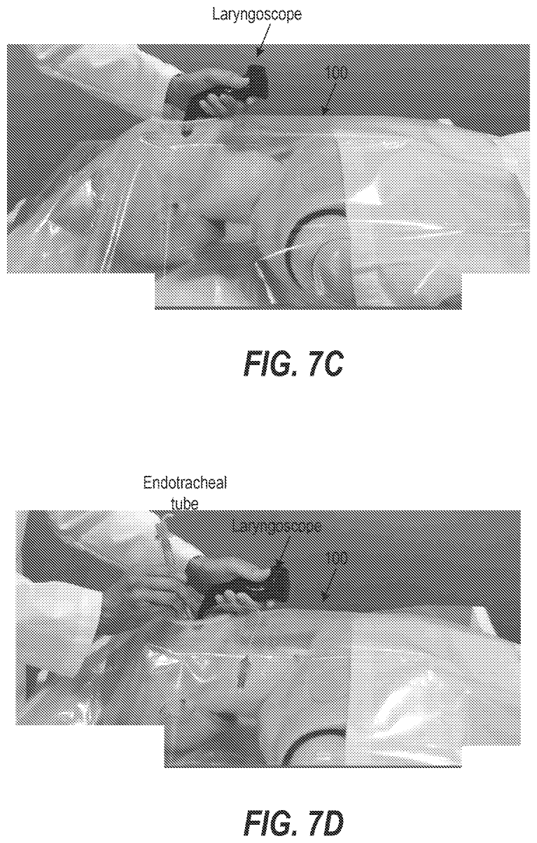

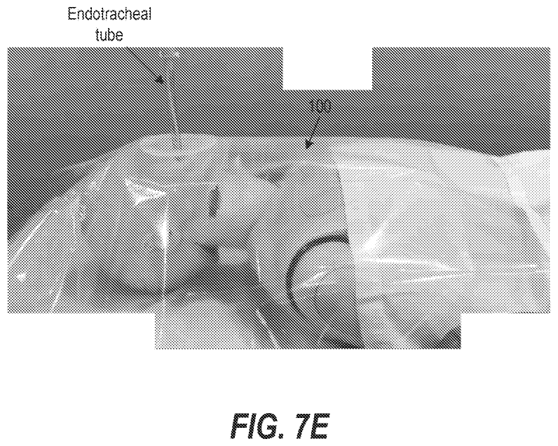

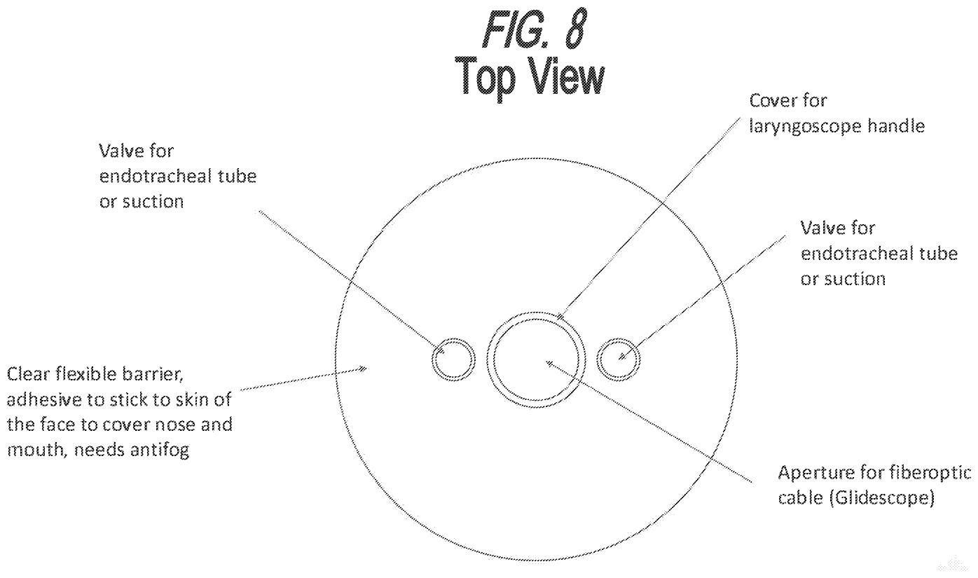

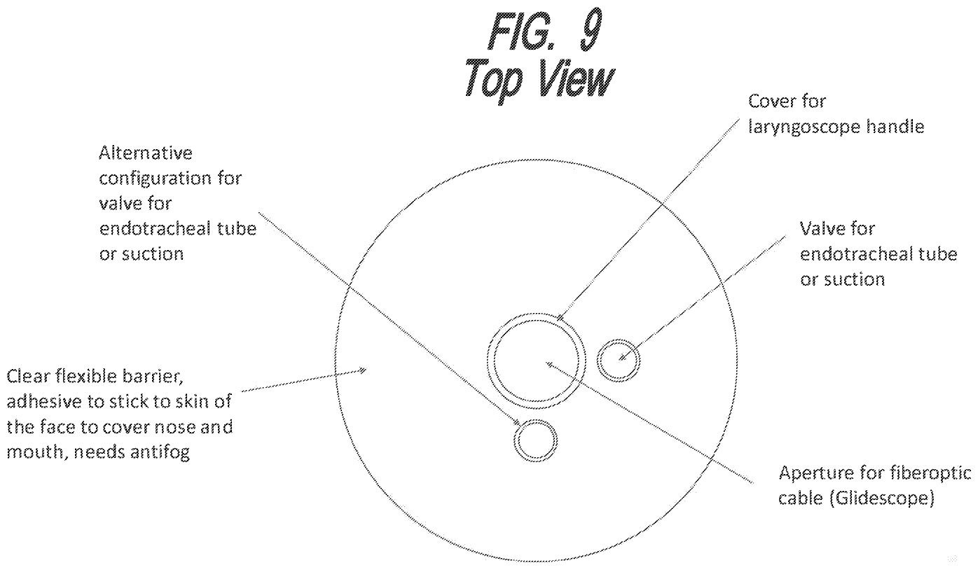

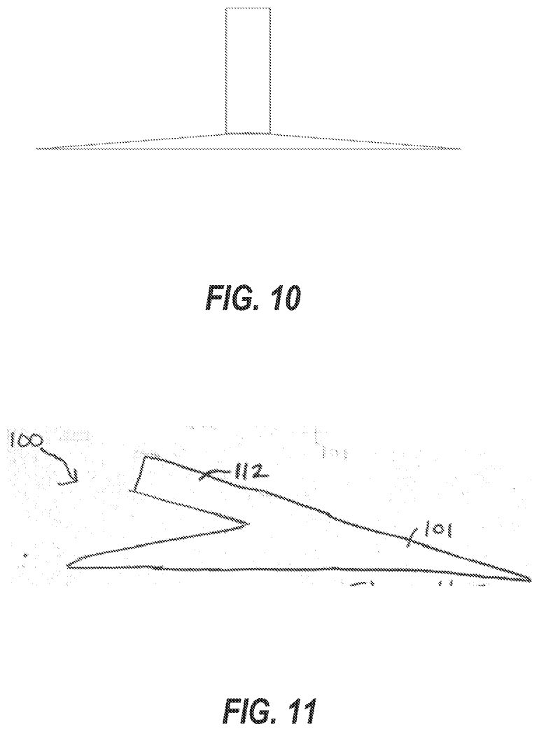

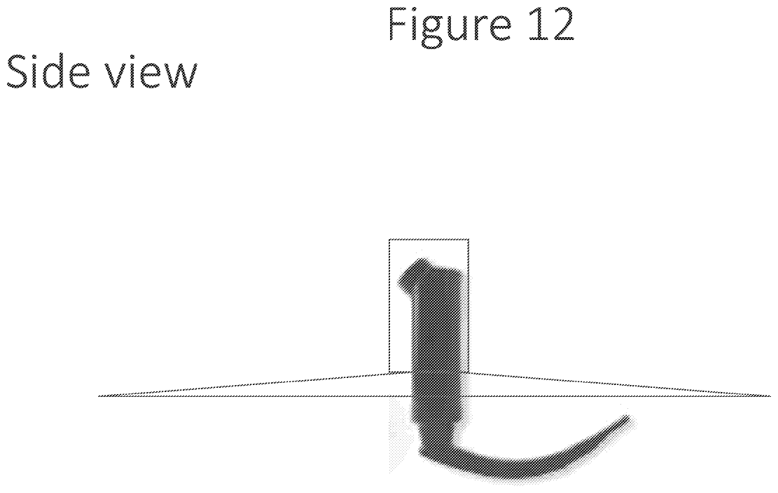

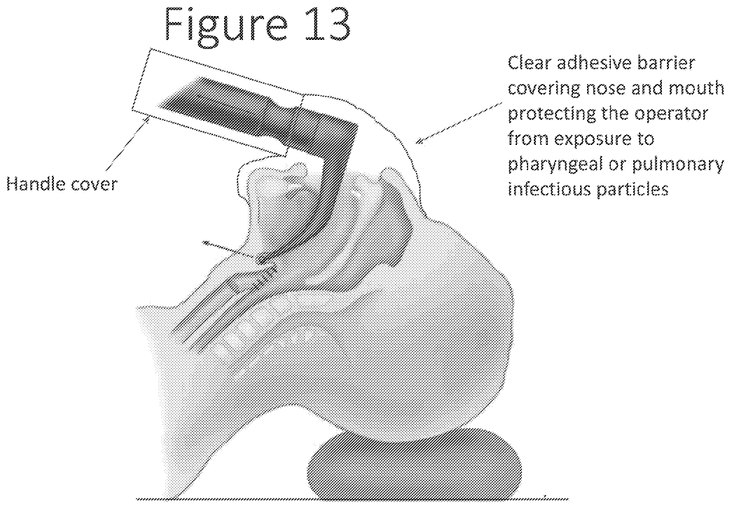

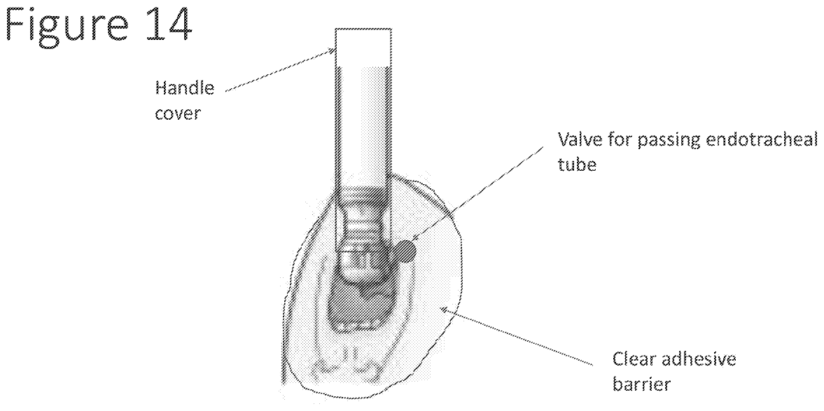

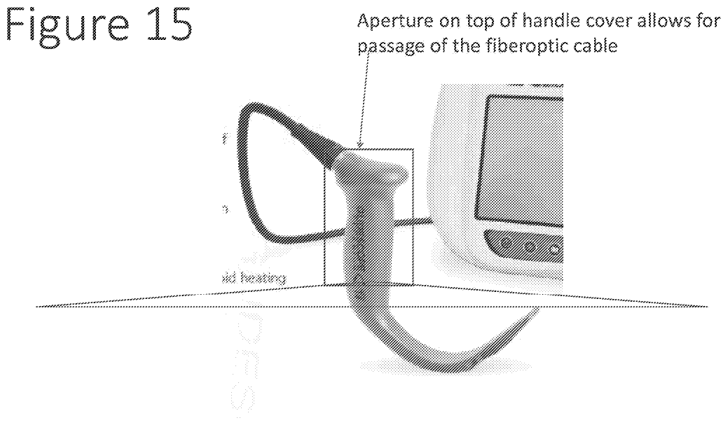

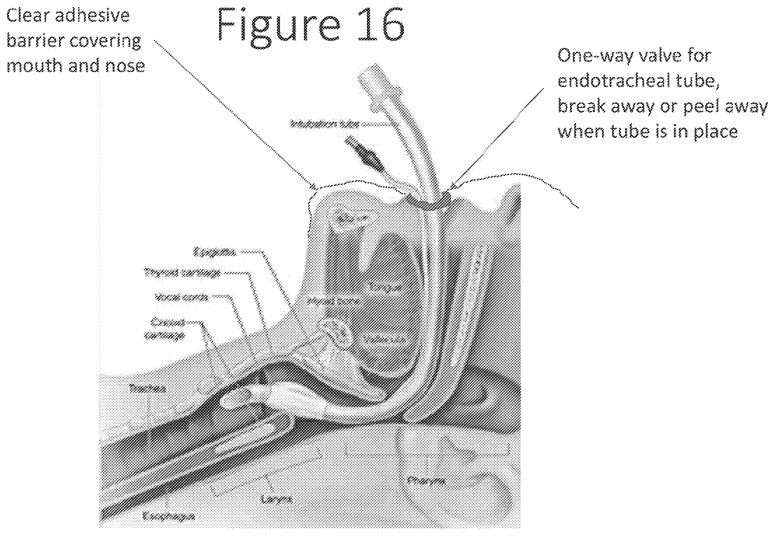

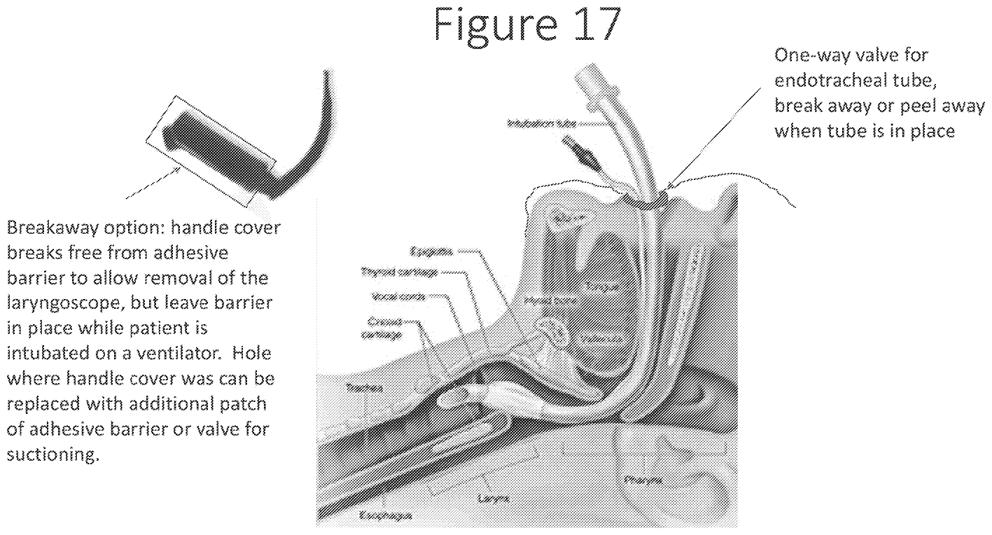

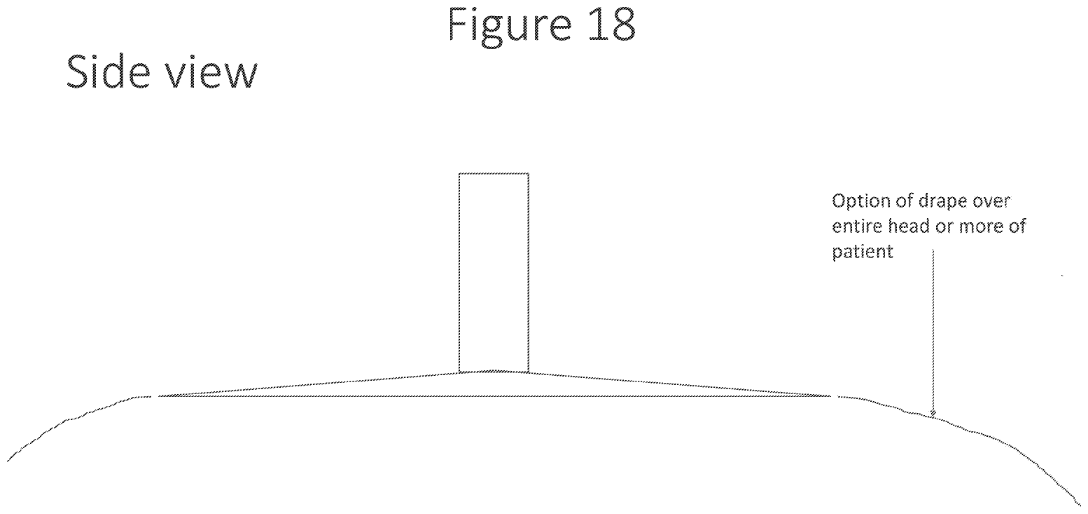

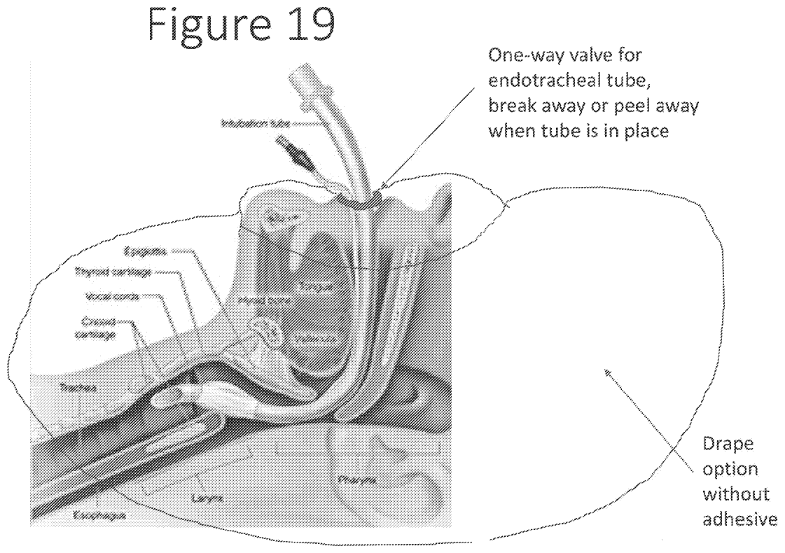

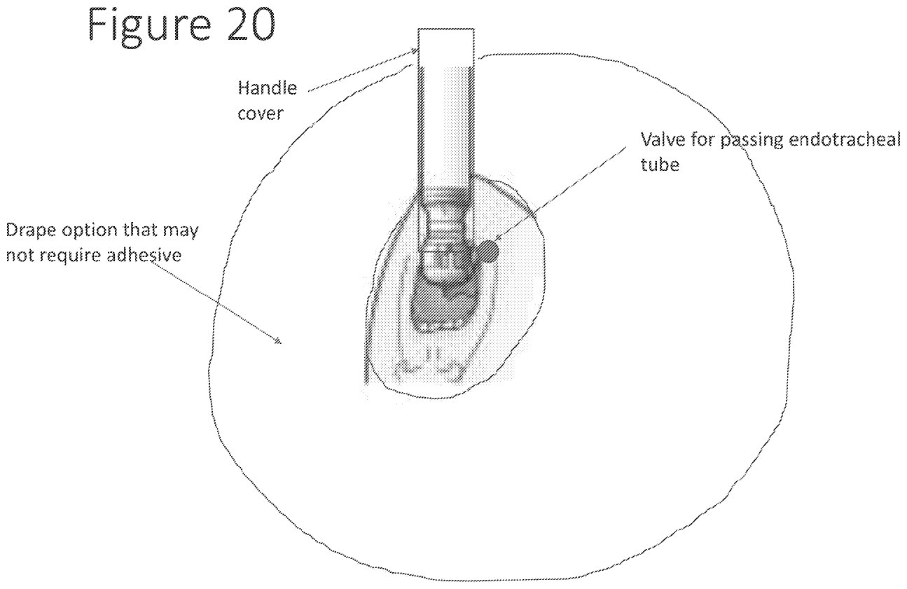

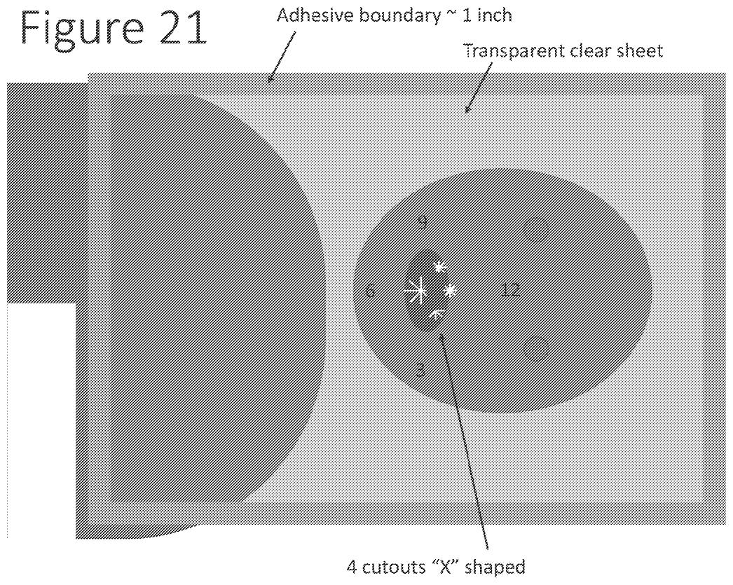

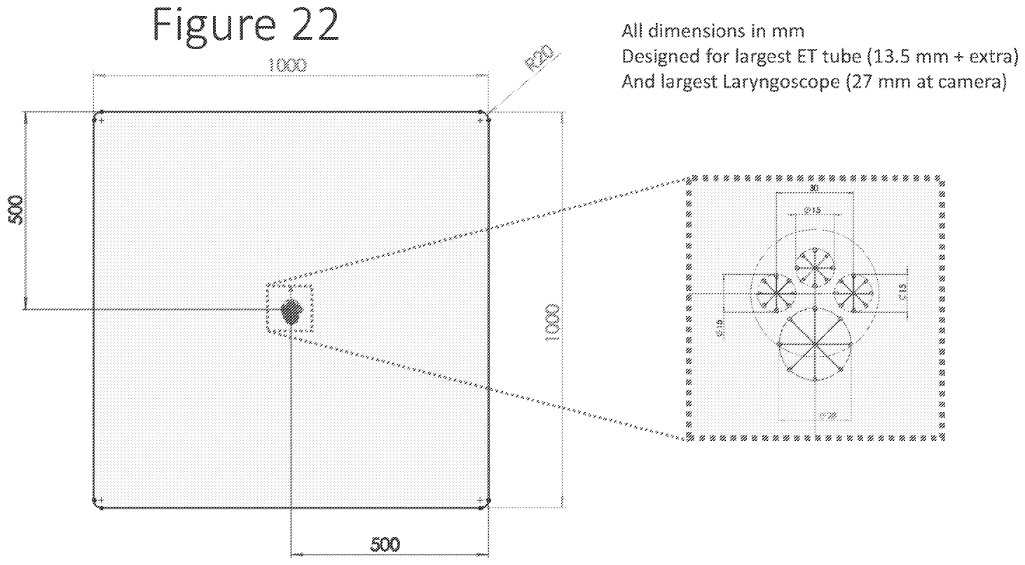

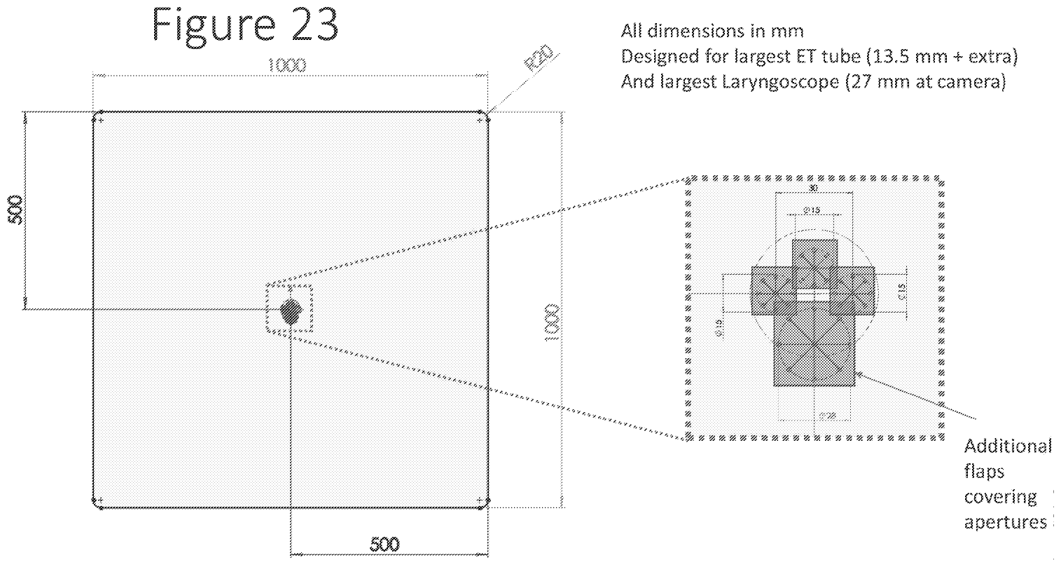

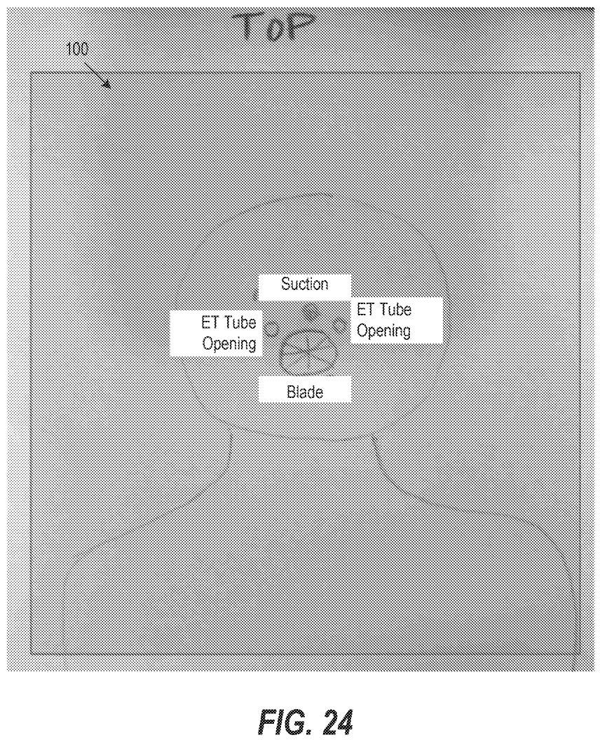

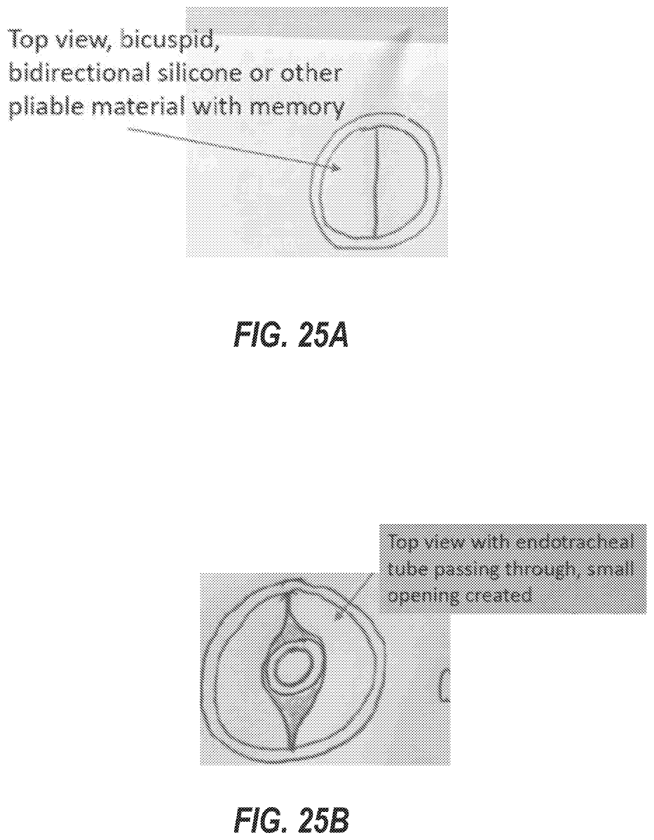

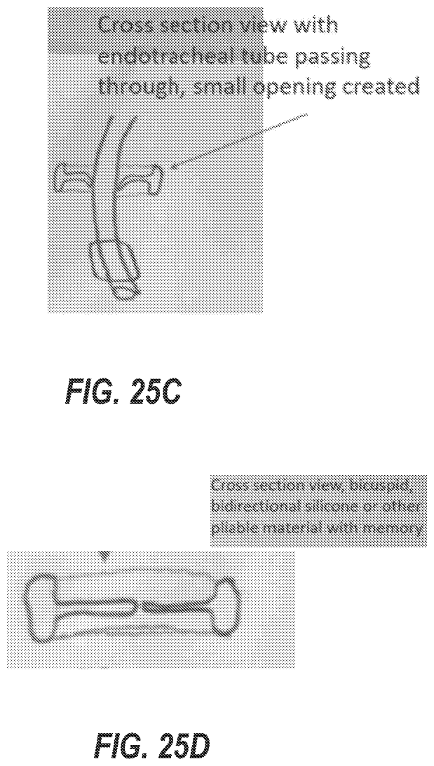

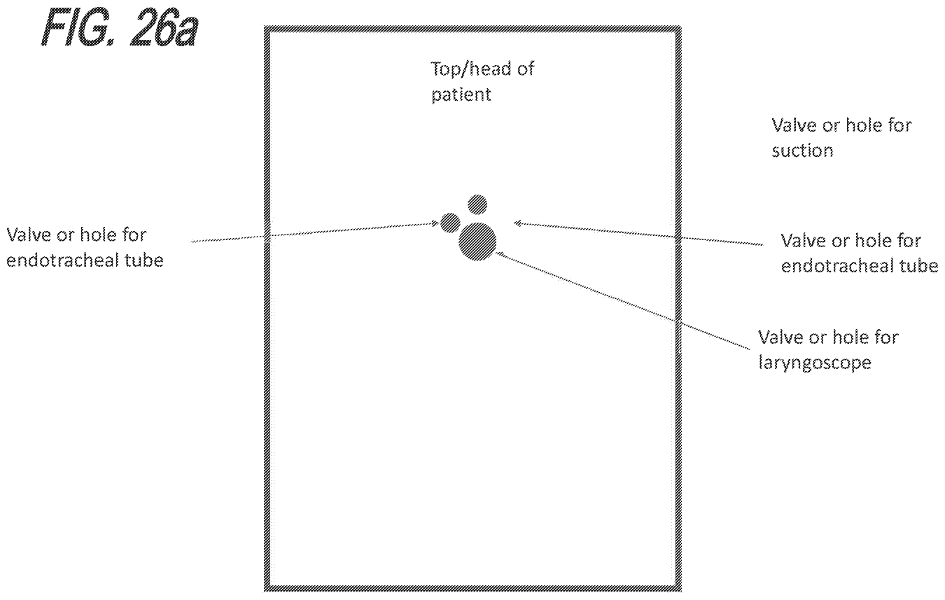

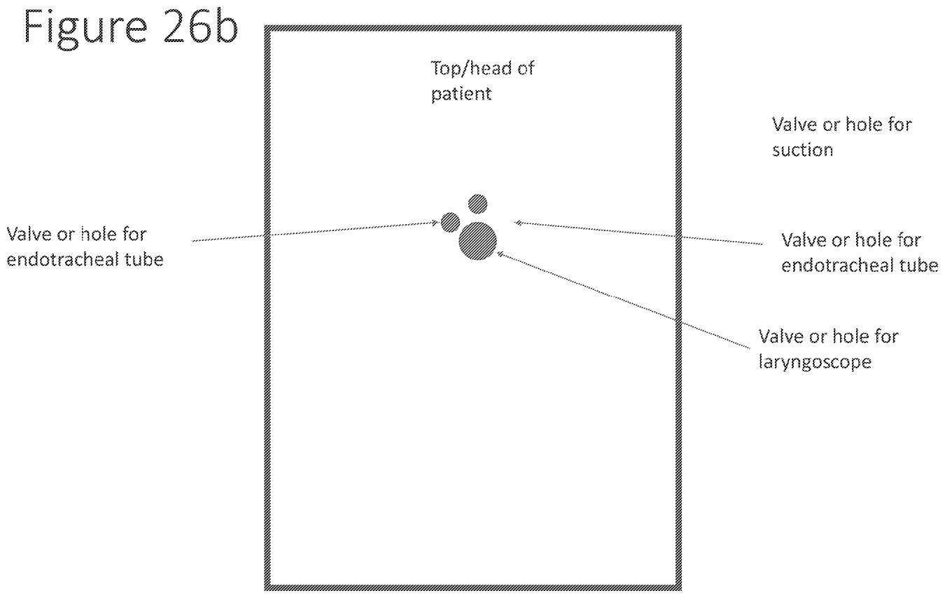

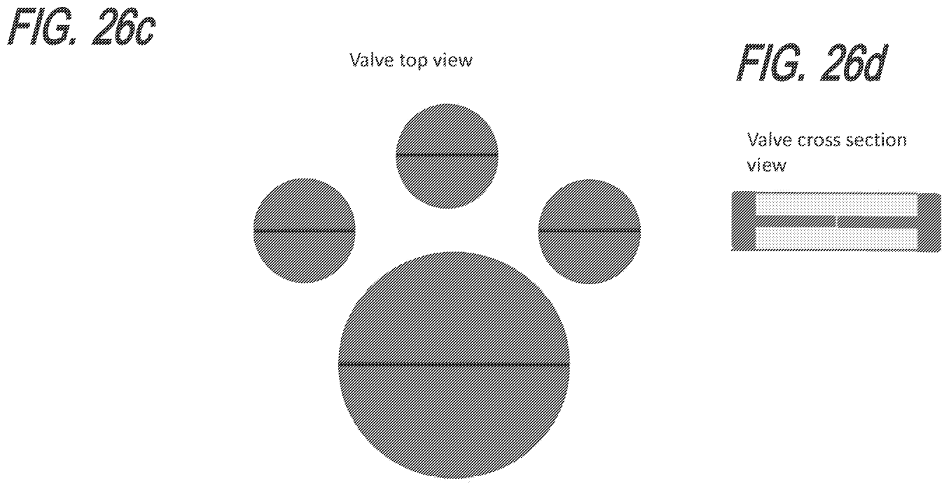

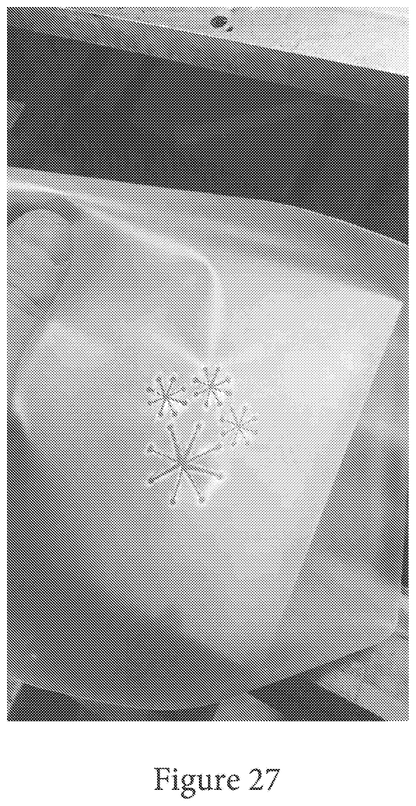

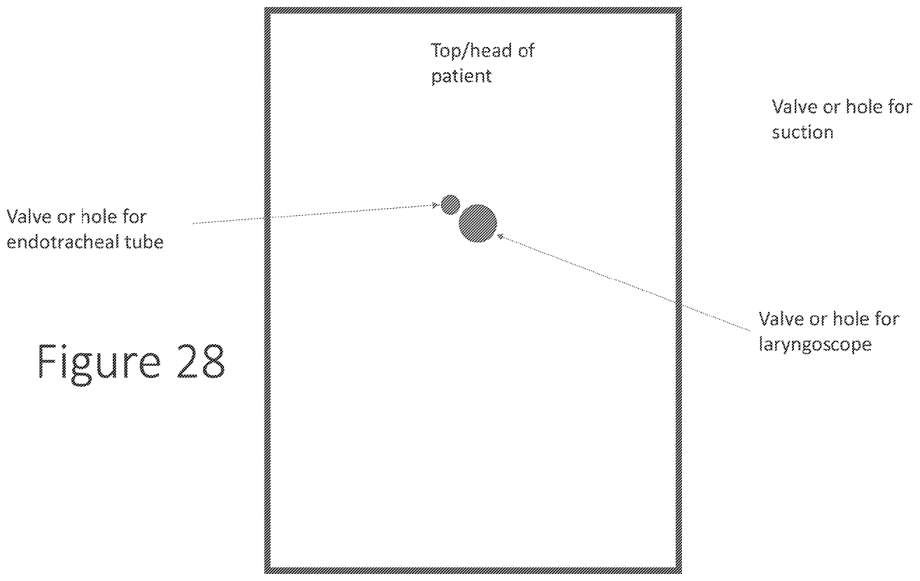

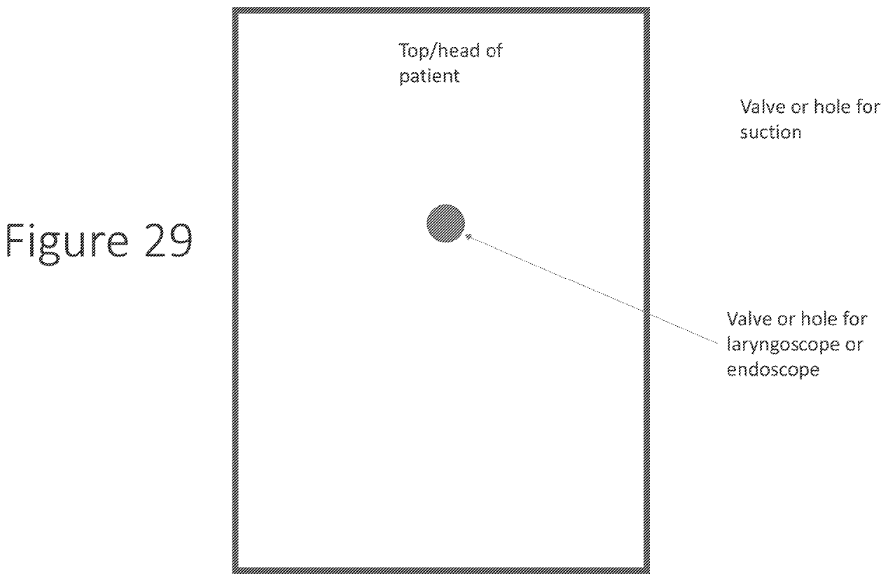

FIG. 7A depicts a barrier configured in accordance with embodiments of the present technology positioned on a patient with an access region aligned with the patient's oral cavity. FIG. 7B is another view of the barrier shown in FIG. 7A. FIGS. 7C-7E depict a barrier configured in accordance with embodiments of the present technology positioned on a patient and receiving multiple instruments therethrough during a medical procedure.

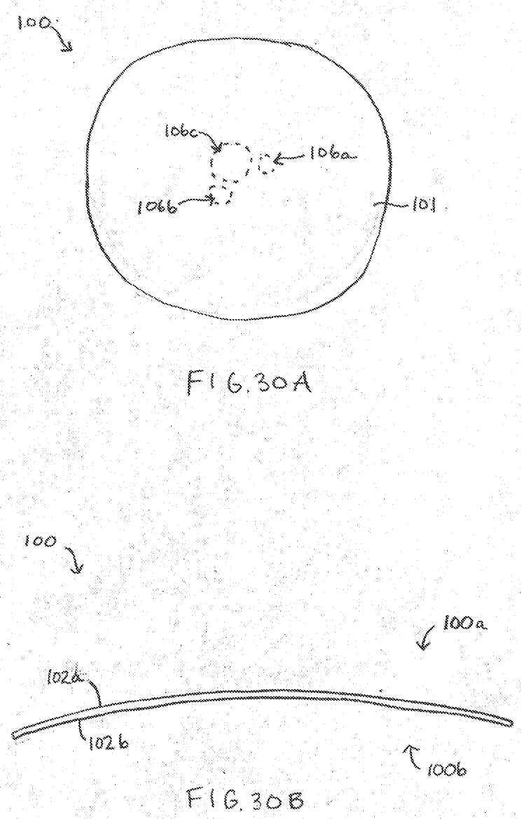

FIGS. 30A and 30B are top and side views, respectively, of a barrier 100 configured in accordance with several embodiments of the present technology. FIG. 31 shows the barrier 100 (in cross-section) positioned on a patient's head prior to any instrumentation being inserted through the barrier 100, and FIG. 32 shows the barrier 100 (in cross-section) with a medical instrument (in this example, a laryngoscope) positioned through the barrier 100 and into the patient's oral cavity and trachea. As shown, the barrier 100 may be configured to be positioned at, on, or over all or a portion of a head of a patient during intubation of the patient. In some embodiments, the barrier 100 may be positioned over additional portions of the patient's body, such as the patient's torso.

The barrier 100 may comprise a flexible member 101 configured to prevent or substantially inhibit dissemination of pathogens from the patient to the surrounding environment. The barrier 100, for example, may be made of silicone, a polymer, and other materials. The barrier 100 may have a first side 100a configured to face towards a healthcare worker during the procedure and a second side 100b configured to face towards the patient during the procedure. Each of the first and second sides 100a, 100b have first and second surfaces 102a, 102b, respectively. All or a portion of the second surface 100b may include an adhesive configured to detachably adhere to the patient's skin or garments, or a nearby structure in the patient's environment (such as the bed, a side rail, etc.) In some embodiments, the second surface 100b does not include any adhesive.

According to several embodiments of the present technology, all or a portion of the barrier 100 is translucent or transparent so that a healthcare worker can see the patient during the procedure.

The barrier 100 may include one or more access regions 106 configured to receive a medical device therethrough, such as an endotracheal access device (such as laryngoscope), an endotracheal tube, a suction device, and/or other devices described herein. As shown in FIGS. 31 and 32, the access regions 106 may be disposed on the barrier 100 such that, when the barrier 100 is positioned on a patient during intubation, the access regions 106 are positioned at or near a mouth of the patient. Each of the access regions 106 may be configured to provide passage of a medical device through the barrier 100 and into an oral cavity of the patient while maintaining a seal around the medical device extending therethrough.

According to some embodiments, for example as shown in FIGS. 30A and 30B, the barrier 100 may include first, second, and third access regions 106a, 106b, 106c. In some embodiments, the barrier 100 may include more or fewer access regions (e.g., a single access region, two access regions, four access regions, etc.). All or some of the access regions 106 may have the same size. In some embodiments, each of the access regions 106 is a different size. According to some embodiments, for example as shown in FIG. 30A, the third access region 106c may be configured to receive a medical instrument having a larger diameter and/or different cross-sectional shape than the first and second access regions 106a, 106b. For example, the first and second access regions 106a, 106b may be configured to receive and endotracheal tube and/or suction device therethrough, and the third access region 106c may be configured to receive an endoscopic device therethrough (such as a laryngoscope).

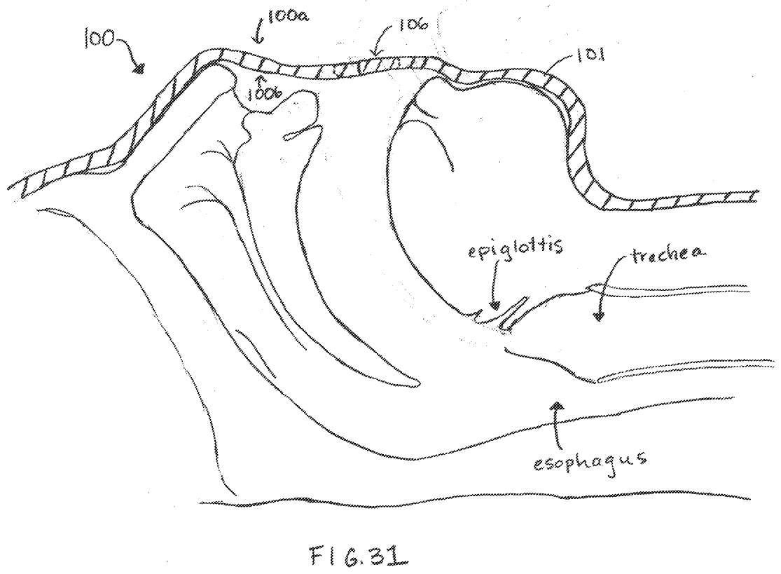

FIG. 33 shows an example access region 106 configured in accordance with the present technology. According to some embodiments, for example as shown in FIG. 33, one or more of the access regions 106 may comprise one or more slits 202 in the flexible member comprising the barrier 100. Each of the slits 202 may meet at an intersection 204. The slits 202 may define flaps or leaflets 200 therebetween that also meet at the intersection 204.

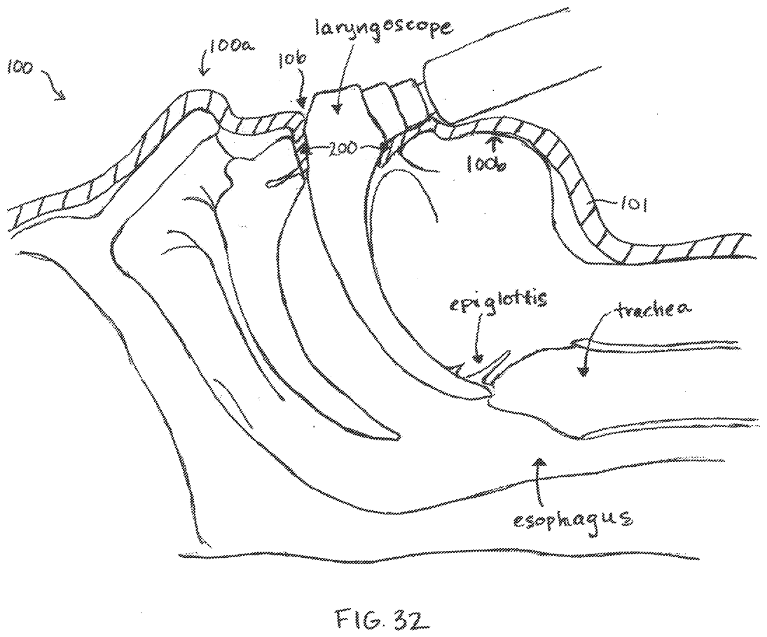

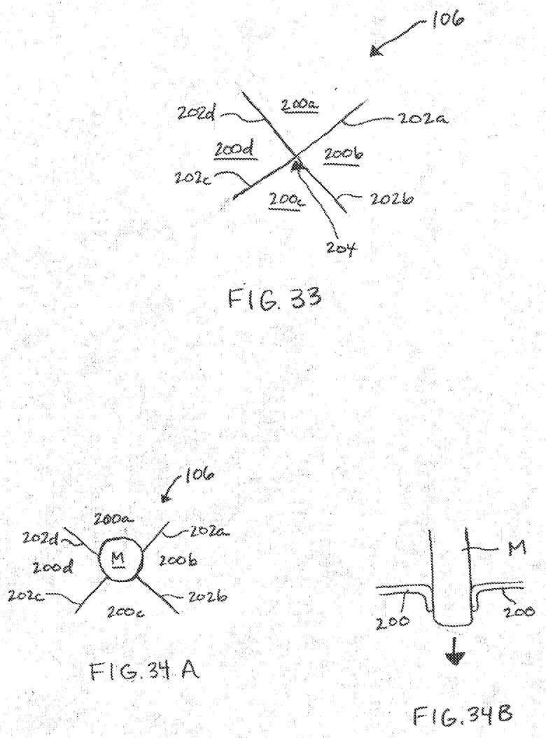

In a resting state, the flaps 200 abut one another and form a closed surface (as shown in FIG. 33. When a downward pressure is exerted on any of the flaps 200, the flaps 200 deform in the direction of the force. For example, FIG. 34A is a top view of a medical instrument M inserted through the access region 106, and FIG. 34B is a cross-sectional side view of the same. As shown, the flaps 200 appose and conform to the outer surface of the medical instrument M while the instrument is received through the access region 106. When the instrument M is removed, the flaps return to their closed, resting position.

The slits 202 provide the operator the ability to move the instrument within the access region to a certain degree without losing contact with the surrounding flaps 200. The slits 202 and flaps 200 are also configured to adapt to changes in the orientation of the barrier during the procedure. For example, if the barrier 100 surrounding access portion 130 is pulled or bent in a particular direction while the instrument is inserted through the access region, the flaps 200 are able to stay apposed and conformed to the instrument.

As described elsewhere herein, different access regions 106 can have the same or different arrangement of slits 202, lengths of slits 202, numbers of slits 202, and/or shapes of slits. The configuration of the access region 106 can be tailored to the medical instrument received therethrough. For example, a medical instrument having a flatter end (such as the blade of a laryngoscope) may have a single slit to provide a better seal around the blade during insertion and withdrawal.

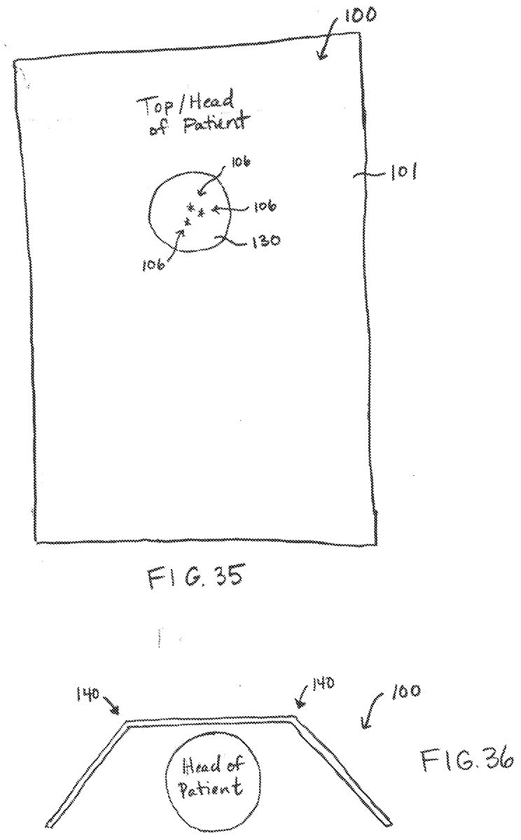

FIG. 35 is a top view of a barrier 100 configured in accordance with several embodiments of the present technology. As shown in FIG. 35, in some embodiments the barrier 100 may comprise an access portion 130 surrounding and forming the access regions 106. The access portion 130 may be stiffer and/or more resilient than the rest of the barrier 100 so that the access regions 106 better conform to the medical instruments inserted therethrough, and so that the access regions 106 return to their closed positions after the instrument is withdrawn. The access portion 130, for example, may comprise a thicker portion of the barrier 100 and/or may be formed of a different material than the rest of the barrier 100. In some embodiments, the access portion 130 comprises a hydrogel, silicone, or other suitable material. The access portion 130 may have the same or different opacity than the remainder of the barrier 100.

In some embodiments, for example as shown in FIG. 36, the barrier 100 may be configured to be loosely draped over the patient. The barrier 100 may include one or more folds 140 or preferential bending lines so that the barrier 100 is predisposed to forming a tent-like structure around the body of the patient.

While many of the embodiments described herein are discussed in the context of endotracheal intubation, the barriers of the present technology may be employed in other applications and/or with other medical instrumentation. For example, the barrier may be configured for use during any procedure of the upper airway or upper gastrointestinal tract requiring instrumentation through the nasal cavity or oral cavity, and may include one or more access regions configured to receive the associated medical instrumentation therethrough.

In some embodiments, the barrier may be configured for use during any intubation, and one or more of the access regions may be configured to receive the associated medical instrumentation therethrough, such as a laryngoscope, an endotracheal tube, a suction device (e.g., a Yankauer device), a laryngeal mask airway (LMA), a Bougie stylet, and/or others.

In some embodiments, the barrier may be configured for use during a bronchoscopy and may have one or more access regions configured to receive the associated instrumentation therethrough, such as a bronchoscope, a bite block, a facemask for oxygenation, and/or others.

In some embodiments, the barrier may be configured for use during an endoscopy (e.g., an esophagoscopy, an esophagogastroduodenoscopy (EGD), an endoscopic retrograde cholangio-pacreatography (ERCP) and may have one or more access regions configured to receive the associated instrumentation therethrough, such as an endoscope, a bite block, a facemask for oxygenation, and/or others.

In some embodiments, the barrier may be configured for use during placement of a nasogastric tube and may have one or more access regions configured to receive the associated instrumentation therethrough.

In some embodiments, the barrier may be configured for use during a fiberoptic nasolaryngoscopy and may have one or more access regions configured to receive the associated instrumentation therethrough.

According to several embodiments, the barrier is configured for use during a biopsy of the nasal cavity, nasopharynx, oral cavity, oropharynx, hypopharynx, larynx, esophagus, stomach, duodenum, biliary tract, and/or pancreas and may have one or more access regions configured to receive the associated instrumentation therethrough.

In many embodiments, the barrier is configured for use during an endoscopic ultrasound and may have one or more access regions configured to receive the associated instrumentation therethrough.

In some embodiments, the barrier is configured for use during placement of gastrostomy tube (PEG, percutaneous endoscopic gastrostomy) and may have one or more access regions configured to receive the associated instrumentation therethrough.

In some embodiments, the barrier is configured for use during aspiration of tonsillar abscess and/or a tonsillectomy) and may have one or more access regions configured to receive the associated instrumentation therethrough.

In some embodiments, the barrier is configured for use during a dental and/or orthodontic procedure, and may have one or more of the access regions configured to receive the associated instrumentation therethrough.

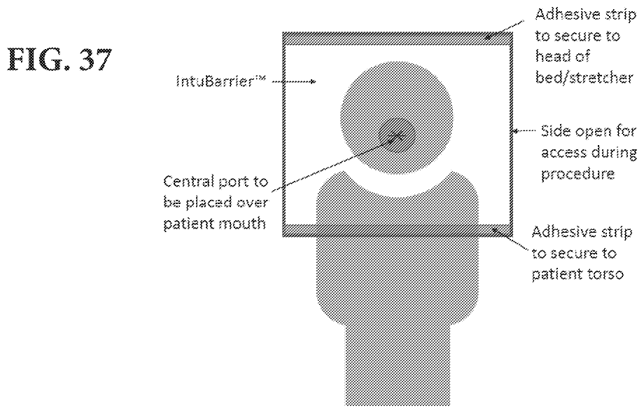

FIG. 37 is a schematic illustration of a barrier configured in accordance with embodiments of the present technology.

### Conclusion

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A device for preventing or substantially inhibiting the dissemination of pathogens from a patient to the surrounding environment during endotracheal intubation of the patient, the device comprising:
a flexible barrier configured to be positioned over all or a portion of the patient's head during the procedure, the barrier comprising a first side configured to face towards the patient during the procedure and a second side configured to face towards a healthcare worker during the procedure, wherein the barrier includes an access region configured to be positioned at or near a mouth of the patient when the barrier is positioned on the patient during the procedure, and wherein the access region is configured to provide passage of a medical device through the barrier and into an oral cavity of the patient while maintaining a physical barrier around the medical device.

2. A device for preventing or substantially inhibiting the dissemination of pathogens from a patient to the surrounding environment during endotracheal intubation of the patient, the device comprising:
a flexible barrier configured to be positioned over all or a portion of the patient's head during the procedure, the barrier comprising a first side configured to face towards the patient during the procedure and a second side configured to face towards a healthcare worker during the procedure, wherein the barrier includes first and second access regions configured to be positioned at or near a mouth of the patient when the barrier is positioned on the patient during the procedure, and wherein each of the first and second access regions are configured to provide passage of a medical device through the barrier and into an oral cavity of the patient while maintaining a physical barrier around the medical device.

3. The device of claim 1, wherein the barrier is sized to cover all or a portion of an upper torso of the patient and a head of the patient.

4. The device of claim 1, further comprising an extension extending from an edge of the barrier wherein the extension is sized to cover all or a portion of an upper torso of the patient and a head of the patient.

5. The device of claim 4, wherein the extension comprises an adhesive at its patient-facing surface configured to adhere to the patient's body.

6. The device of claim 2, wherein the first and/or second access regions are configured to receive at least one of an endotracheal tube, a laryngoscope, an endoscope, or a suction device therethrough.

7. The device of claim 2, wherein the first and/or second access regions comprise slits in the barrier.

8. The device of claim 2, wherein the first and/or second access regions comprise a single slit in the barrier.

9. The device of claim 2, wherein the first and/or second access regions comprise a plurality of slits arranged in an “x”, star, or pinwheel shape.

10. The device of claim 2, wherein the first and/or second access regions comprise a plurality of linear and/or curved slits radiating outwardly from a central point.

11. The device of claim 2, wherein the first and/or second access regions include a valve.

12. The device of claim 2, wherein the first and/or second access regions include a bicuspid valve.

13. The device of claim 2, wherein the first and/or second access regions include a valve configured to open in response to insertion of an endotracheal tube or suction device, and close once the endotracheal tube or suction device is withdrawn.

14. The device of claim 2, wherein the first and/or second access regions include a spring-activated hinge valve.

15. The device of claim 2, wherein the barrier further comprises a third access region configured to receive at least one of an endotracheal tube and/or a suction device therethrough.

16. The device of claim 15, wherein the barrier further comprises a fourth access region configured to receive a medical instrument therethrough.

17. The device of claim 1, wherein the barrier is disposable.

18. The device of claim 1, wherein the barrier is configured for single use.

19. The device of claim 1, wherein at least a portion of the barrier is translucent.

20. The device of claim 1, wherein at least a portion of the barrier is transparent.

21. The device of claim 1, wherein all of the barrier is translucent.

22. The device of claim 1, wherein all of the barrier is transparent.

23. The device of claim 1, wherein the barrier comprises an antifog coating.

24. The device of claim 1, wherein the barrier comprises an adhesive along all or a portion of a surface of the first side, the adhesive configured to (a) detachably adhere to the patient's skin and/or (b) detachably adhere to a surface in the vicinity of the patient.

25. The device of claim 1, wherein the barrier comprises an adhesive along selected portions of a surface of the first side, wherein, wherein the barrier is positioned on the patient's head during the procedure, the selected portions correspond to one or more of the patient's nose, the patient's cheeks, and/or a portion of the patient's head inferior to a chin of the patient.

26. The device of claim 1, wherein the barrier comprises a reinforced portion at the access region, wherein the reinforced portion has a thickness greater than a thickness of the surrounding portions of the barrier.

27. The device of claim 1, wherein the barrier comprises a reinforced portion at the access region, wherein the reinforced portion is less flexible than the rest of the barrier.
